(19) United States
(12) Patent Application Publication
(10) Pub. No.: US 2026/0069614 A1
(43) Pub. Date: Mar. 12, 2026

(54) NOVEL PARENTERAL FORMULATIONS OF CANNABIDIOL

(71) Applicant: LEIUTIS PHARMACEUTICALS LLP, Hyderabad (IN)

(72) Inventors: Chandrashekhar KOCHERLAKOTA, Hyderabad (IN); Nagaraju BANDA, Hyderabad (IN); Arjun NARALA, Warangal (IN); Srividya RAMREDDY, Hyderabad (IN); Naga Udaya Sankar PULLAGURA, Hyderabad (IN); Srinath AKULA, Hyderabad (IN)

(21) Appl. No.: 19/107,655

(22) PCT Filed: Aug. 30, 2023
(86) PCT No.: PCT/IB2023/058549

§ 371 Date: Feb. 28, 2025

(30) Foreign Application Priority Data
Aug. 30, 2022 (IN) 202241049537

(51) Int. Cl. A61K 31/00; A61K 9/107; A61K 31/167; A61K 31/195; A61K 31/197; A61K 31/381; A61K 31/496; A61K 31/573; A61K 47/02; A61K 47/10; A61K 47/12; A61K 47/14; A61K 47/24

(52) U.S. Cl. CPC A61K 31/658; A61K 9/107; A61K 31/167; A61K 31/195; A61K 31/197; A61K 31/381; A61K 31/496; A61K 31/573; A61K 47/02; A61K 47/10; A61K 47/12; A61K 47/14; A61K 47/24

(57) ABSTRACT

The present invention relates to parenteral emulsion formulations of cannabidiol comprising synthetic cannabidiol (CBD), omega 3 components and other pharmaceutically acceptable excipients. The invention further relates to parenteral emulsion formulations comprising synthetic cannabidiol in combination with one or more active agents, omega 3 components, and other pharmaceutically acceptable excipients. The formulations are used to treat acute and chronic pain, inflammatory conditions, hepatic, cardiovascular disorders, respiratory conditions, immune disorders, and CNS disorders.

### FIELD OF THE INVENTION

The present invention relates to parenteral emulsion formulations of cannabidiol comprising synthetic cannabidiol (CBD), omega 3 components and other pharmaceutically acceptable excipients. The invention further relates to parenteral emulsion formulations comprising synthetic cannabidiol in combination with one or more active agents, omega 3 components, and other pharmaceutically acceptable excipients. The formulations are used to treat acute and chronic pain, inflammatory conditions, hepatic, cardiovascular disorders, respiratory conditions, immune disorders, and CNS disorders.

### BACKGROUND OF THE INVENTION

Cannabidiol (CBD) is a phytocannabinoid present in cannabis plant and is one among 113 identified cannabinoids. CBD has a complex pharmacological mechanism and acts as an anti-psychotic, analgesic, anti-convulsant, muscle-relaxant, anxiolytic, anti-inflammatory, anti-neoplastic, antiproliferative, and an anti-angiogenic. At lower doses, it has physiological effects that promote and maintain health, including antioxidative, anti-inflammatory, and neuroprotective effects. CBD is considered more effective than vitamin C and E as a neuroprotective antioxidant and can also be used to treat skin conditions such as acne.

In the United States, CBD is available as Epidiolex®, an oral solution (100 mg/mL) for treating seizures associated with Lennox-Gastaut syndrome, Dravet syndrome, or tuberous sclerosis complex. CBD is highly lipophilic and undergoes extensive first-pass metabolism resulting in low bioavailability.

Omega-3 components are highly unsaturated fatty acids found in sea foods that are essential for the functioning of cells and organs. Of particular importance are omega 3 fatty acids such as docosahexaenoic acid (DHA), docosapentaenoic acid (DPA), and eicosapentaenoic acid, omega 3 ethyl esters, non-phosphate containing glycerolipids such as omega 3 triglycerides, diacylglycerol, monoacylglycerol, phospholipids. These are useful in treating various conditions related to cardiovascular, pulmonary, neurological, immune and endocrine systems. 03 components converted into bioactive lipid metabolites forming endocannabinoid system in the body. These endocannabinoids mimic the functions of cannabinoids. Therefore, CBD when combined with O3 components may result in better health benefits.

International publication No. WO2022038528 (A1) to Chandrashekhar et al., discloses nano lipid carrier system of cannabidiol for oral administration. These cannabidiol formulations comprise active ingredients, low phase transition substance, pegylated lipid and other excipients dispersed in water or aqueous medium. These systems have a hydrodynamic diameter (HDD) of less than about 1000 nm in aqueous media.

U.S publication No. US2021186870 (A1) to Garabagi Freydoun et al., discloses cannabinoid formulations for oral administration comprising one or more cannabinoids and a lipid carrier comprising oils.

International publication No. WO2016147186 (A1) to Sinai Alon et al., discloses emulsion compositions comprising of tetrahydrocannabinol (THC), cannabidiol (CBD) or derivatives thereof, phospholipids, an oily fraction comprising about 50% cannabinoids. These emulsions are intended for topical, oral or nasal administration and are not suitable for parenteral administration.

U.S. Pat. No. 11,229,612 (B2) to Stephen et al., discloses parenteral formulations of cannabinoid, comprising of an isotonic agent, a surfactant, bulking agent, solvent and one or more stability enhancers. These formulations are spray dried or lyophilized and therefore need to be reconstituted with a suitable diluent before administration.

U.S publication No.: US2022193004 (A1) to Jackson et al., discloses parenteral formulations comprising at least one cannabinoid from natural or synthetic source, at least one surfactant, at least two antioxidants, at least one chelating agent and buffering agent.

International publication No.: WO2019140325 (A1) to Christopher et al., discloses inhalation and injectable emulsions formulations of cannabinoids prepared using at least one co-solvent ranging from 0.5% w/v to 50% w/v (ethanol) and at least one surfactant.

International publication No.: WO2019094625 (A1) to Alexander et al., discloses natural or synthetic cannabidiol formulations alone or in combination with anti-epileptic drugs prepared using various nano lipid carrier systems, which require robust and time taking techniques to formulate.

International publication No. WO2021077211 (A1) to Bolton et al., discloses method of treating heart related conditions using cannabidiol. These parenteral formulations are prepared using various solvents including fatty acids, triglycerides, ethanol, and lipids. Specific exemplified formulations are not covered.

Most of the CBD used in the art and in commercial preparations is of natural origin and often may have high levels of THC and other undesirable plant related substances. THC causes many undesirable effects and patients may experience euphoria and anxiolysis as well as enhanced sensory perceptions. Higher doses of THC are associated with anxiety, panic and disorientation.

The present invention presents a parenteral emulsion formulation of CBD with less than 1% THC. The emulsion formulations may additionally comprise one or more active agents and may be used for treating acute and chronic pain, inflammation, CNS and immune disorders.

### SUMMARY OF THE INVENTION

One aspect of the invention is to provide a parenteral emulsion formulation of synthetic cannabidiol, wherein the formulation has less than 1.0% tetrahydrocannabinol (THC).

Another aspect of the invention is to provide a parenteral emulsion formulation of synthetic cannabidiol, wherein the formulation has less than 0.5% tetrahydrocannabinol (THC).

Yet another aspect of the invention is to provide a parenteral emulsion formulation comprising synthetic cannabidiol, omega 3 component and other pharmaceutically acceptable excipients.

Yet another aspect of the invention is to provide a parenteral emulsion formulation comprising synthetic cannabidiol and omega 3 fatty acids or omega 3 fatty acid ethyl ester or omega 3 triglycerides and other pharmaceutically acceptable excipients.

Another aspect of the invention is to provide a parenteral emulsion formulation comprising synthetic cannabidiol and omega 3 fatty acids, or omega 3 fatty acid ethyl ester or omega 3 triglycerides, wherein the concentration of CBD ranges from about 0.05 mg/ml to about 250 mg/ml and of omega 3 component ranges from about 1 mg/ml to about 500 mg/ml.

Yet another aspect of the invention is to provide a parenteral emulsion formulation comprising synthetic cannabidiol in combination with other active ingredients, omega 3 component and other pharmaceutically acceptable excipients.

### DETAILED DESCRIPTION OF THE INVENTION

The term “CBD” described in the present invention refers to synthetic cannabidiol or its derivatives. The content of CBD in the formulation may range from about 0.005% w/v to 25% w/v.

The term “parenteral administration” refers to administration through subcutaneous injection, intramuscular injection, intra-articular injection, intravenous injection, intrathecal injection or joint injection or infiltration injection.

“O3 component or omega 3 component” in the context of the invention refers to (i) the highly unsaturated fatty acids (the omega 3 fatty acids) found in sea foods or (ii) the omega 3 ethyl ester found in fish oil or (iii) the omega 3 triglycerides or (iv) alpha-lipoic acid and its derivatives. The formulation of the present invention preferably comprises one or more omega 3 components including docosahexaenoic acid (DHA), docosapentaenoic acid (DPA), and eicosapentaenoic acid (EPA). In the event the formulation comprises more than one O3 component, the ratio of these may vary.

The present invention provides a parenteral emulsion formulation of cannabidiol comprising synthetic CBD, omega 3 component in the form of omega 3 fatty acids or omega 3 ethyl ester or omega 3 triglycerides and other pharmaceutically acceptable excipients, wherein the composition comprises less than 1% THC. Preferably the formulation has less than 0.5% THC and more preferably less than 0.3% THC. It is most preferable that the formulation does not contain a detectable limit of THC as determined by standard analytical techniques.

The parenteral emulsion formulation comprises an oil phase and an aqueous phase. The oil or lipid phase comprises cannabidiol dispersed/dissolved in the oil or the lipid vehicle. The oil phase may optionally comprise polymers, co-solvents, surfactants and stabilizers. The aqueous phase comprises of one or more aqueous vehicles and optional cosolvents, surfactants, stabilisers, buffers and isotonicity agents. The oil phase ranges from about 5% w/v to about 40% w/v and the aqueous phase ranges from about 25% w/v to about 95% w/v in the total formulation.

The emulsion formulation preferably comprises globules with a D₉₀ less than 550 nm and a hydrodynamic diameter (HDD) less than 250 nm. More preferably the D₉₀ ranges from about 75 nm to 450 nm and HDD ranges from about 50 nm to 300 nm. It is advantageous to have a formulation with the said globule size as it minimises the irritation and discomfort to the patient.

The concentration of cannabidiol ranges from about 0.05 mg/ml to about 250 mg/ml or about 0.005% to 25% (w/v) in the formulation. In the oil phase, CBD constitutes about 2% w/v to 60% w/v.

The oil phase in the emulsion formulation comprises one or more omega 3 components selected from the group comprising omega 3 fatty acids such as docosahexaenoic acid (DHA), docosapentaenoic acid (DPA), and eicosapentaenoic acid (EPA) or omega 3 ethyl esters or omega 3 triglycerides. The concentration of O3FA or the ethyl esters may range from about 0.1% w/v to about 50% w/v in the formulation. The content of omega 3 component may vary from about 1 mg/ml to about 500 mg/ml.

The oil phase may additionally comprise one or more of medium chain triglycerides USP/NF (Labrafac Lipophile WL 1349), medium chain triglyceride oil, sunflower oil, triacetin, coconut oil, grape seed oil, oleic acid, decanoic acid, free fatty acids and their derivatives, and triglycerides of free fatty acids, vegetable/plant and animal derived oils, phospholipid derivatives of fatty acids.

The aqueous phase comprises one or more pharmaceutically acceptable excipients selected from the group comprising co-solvents, buffers, surfactants, stabilizing agents, preservatives, chelating agents, tonicity modifiers. The aqueous phase may constitute from about 25% w/v to 95% w/v based on the total weight of the formulation.

The pharmaceutical formulation of the present invention may additionally comprise inactive ingredient(s) selected from the group comprising polymers, solvents, co-solvents, buffers, surfactants, stabilizers, preservatives, chelating agents and tonicity modifiers.

Co-solvent(s) may range from about 0 to 25% (w/v) and may be selected from, but not limited to, ethanol, N-methyl-2-pyrrolidone (NMP), tertiary butyl alcohol (TBA), glycerol, propylene glycol, polyethylene glycol, Transcutol HP, Gelucire® 50/13 (stearoyl polyoxylglycerides) etc.

Buffer(s) or pH adjusting agent(s) described in the present invention are selected from L-histidine, L-arginine, tris base, meglumine, glycine, sodium succinate, diethanolamine, aspartic acid, glutamic acid, alanine, sodium acetate, sodium ascorbate, sodium citrate, citric acid, succinic acid, triethanolamine, boric acid, sodium hydroxide, sodium hydrogen carbonate and the like.

Stabilizer(s) may range from about 0.5 to 5% (w/v) and are selected from, but not limited to, antioxidants like vitamin E acetate, ascorbyl palmitate, ascorbic acid, monothioglycerol (MTG), citric acid, tartaric acid etc and chelating agents like 1,4,7,10-tetraazacyclododecane-1,4,7,10-tetra acetic acid (DOTA), disodium EDTA etc.

Tonicity agent(s) or isotonic agent(s) may range from about 0.5 to 10% (w/v) and may be selected from but not limited to dextrose, mannitol, glycerol and sodium chloride.

Surfactant(s) may range from about 0.5 to 15% (w/v) and are selected from, but not limited to amphiphilic surfactants, lipophilic surfactants comprising like alpha lecithin, cholesterol oleate; phospholipids like soy lecithin, egg lecithin (LIPOID E 80 S/Egg Lip 80 W), hydrogenated soy lecithin, alpha lecithin; modified phospholipids derivatives of dimyristoyl phosphatidylcholine (DMPC), 1,2-dipalmitoyl-rac-glycero-3-phosphocholine (DPPC), dimyristoyl phosphatidyl ethanolamine (DMPE), 1,2-bis(diphenylphosphino) ethane (DPPE), dimyristoylphosphatidylglycerol (DMPG), dipalmitoyl phosphatidylglycerol (DPPG), 1,2-distearoyl-sn-glycero-3-phosphoryl ethanolamine (DSPE), 1,2-dioleoyl-sn-glycero-3-phosphoethanolamine (DOPE), hydrophilic surfactants like poloxamer-188, sodium oleate, sodium cholate, ethylene oxide/propylene oxide copolymers like poloxamer 182, poloxamer 407 and poloxamine 908, poloxamer-188, non-ionic surfactants like polysorbate 20, polysorbate 60, polysorbate 80 (Tween 80), Span-20, Span-60 and Span-80, Cremophor EL, anionic bile salts sodium cholate, sodium glycocholate, sodium tauro cholate, sodium tauro deoxy cholate and sucrose esters of fatty acids (ex: sucrose laurate, sucrose oleate, sucrose palmitate, sucrose stearate and the like), Kolliphor RH40, pegylated lipids such as N-(Carbonyl-methoxypolyethylenglycol-2000)-1,2-distearoyl-sn-glycero-3-phospho ethanolamine, MPEG-2000-DSPE, Na-salt (Lipoid PE 18:0/18:0-PEG 2000) (PEG-PE) etc.

In one embodiment the parenteral emulsion formulation comprises (a) an oil phase comprising (i) cannabidiol dispersed or dissolved in the oil phase, (ii) a omega 3 component selected from omega 3 fatty acids, omega 3 ethyl esters and omega 3 triglycerides and (b) an aqueous phase and appropriate pharmaceutically acceptable excipients in the oil phase and aqueous phase.

The formulations of the present invention are used to treat various conditions including acute and chronic pain, neuralgia, inflammatory conditions, hepatic, cardiovascular disorders, respiratory conditions, immune disorders, and CNS disorders such as anxiety, depression, and epilepsy.

The parenteral emulsion formulation contains less than 1% THC, preferably less than 0.5% THC and more preferably less than 0.3% THC. It is most preferable that the formulation does not contain a detectable limit of THC as determined by standard analytical techniques.

The formulations of present invention retained at least 90% of CBD even after storage for more than 12 months at room temperature.

The formulations of present invention have a pH ranging from 2.5-9.75.

The formulations of present invention were tested for stability and the details are as follows:

TABLE 1
Stability data

| Examples | Example 1 | | F9 | | F10 | | F11 | |
|---|---|---|---|---|---|---|---|---|
| CBD Conc. | 8 mg/mL | | 6 | mg/mL | 2 | mg/mL | 5 | mg/mL |
| Temperature | RT | | | | | | | |
| Time point | Initial | 15 | 3 | Months | 6 | Months | 3 | Months |
| | | Months | | | | | | |

| Description | Milky white solution | | | | |
|---|---|---|---|---|---|
| pH | 4.01 | 4.06 | 4.35 | 4.44 | 3.61 |
| *Assay (%) | 98.75 | 91.25 | 98.33 | 110 | 100 |
| THC | ND | ND | ND | ND | ND |

*Assay: % of drug retained

In another embodiment the parenteral emulsion formulation comprises (a) an oil phase comprising (i) cannabidiol dispersed or dissolved in the oil phase, (ii) a omega 3 component selected from omega 3 fatty acids, omega 3 ethyl esters and omega 3 triglycerides (iii) one or more surfactants and (b) an aqueous phase and appropriate pharmaceutically acceptable excipients in the oil phase and aqueous phase.

Yet another embodiment relates to a parenteral emulsion formulation of cannabidiol with (a) an oil phase comprising (i) cannabidiol dispersed or dissolved in the oil phase, (ii) an omega 3 ethyl ester and (iii) egg lecithin (iv) pegylated lipid and (b) an aqueous phase and appropriate pharmaceutically acceptable excipients in the oil phase and aqueous phase.

Parenteral formulations may be of great advantage in treating acute and chronic conditions, conditions where localised action is desired, conditions where immediate therapeutic effect is required or where there are undesirable side effects in other routes of administration. This formulation may be useful in treating various disorders like acute and chronic pain, inflammatory, immune and CNS disorders. The formulation does not exhibit any adverse psychological effect or cause a psychological disorder in the patient.

The present invention may also be adapted to be administered in combination with at least one pharmaceutically active agent. Such active agents may be selected from the group comprising non-steroidal anti-inflammatory drugs (NSAID), anti-histamines, anti-fibrotic agents, anti-epileptic drugs, anxiolytics, antidepressants, anti-psychotic drugs, opiate antagonists, psychoactive agents, analgesics, immunosuppressants, anti-insomnia drugs, neuroprotective agents, leukotriene receptor antagonists (LTRAs), lipid-regulating agents, HMG-COA reductase inhibitors etc. The dose of the individual active agents will depend on the condition to be treated.

The present invention formulations further comprise of drugs selected from any class of BCS classification and this nomenclature is well understood by any person skilled in the art. The formulations of present invention includes drugs of class 1 such as pregabalin, melatonin, ketorolac tromethamine, tramadol, tapentadol, drugs of class 2 such as duloxetine, naproxen, atorvastatin, rosuvastatin, montelukast, brexpiprazole, lurasidone, prochlorperazine edisylate, bilastine, drugs of class 3 such as gabapentin, acetaminophen, tofacitinib, cetirizine, naloxone, morphine and drugs of class 4 such as naltrexone, allopregnanolone.

In one embodiment, the parenteral emulsion formulation comprises (a) an oil phase comprising (i) cannabidiol dispersed or dissolved in the oil phase, (ii) an omega 3 ethyl ester and (b) an aqueous phase comprising gabapentin and appropriate pharmaceutically acceptable excipients in the oil phase and aqueous phase.

In another embodiment, the parenteral emulsion formulation comprises (a) an oil phase comprising (i) cannabidiol dispersed or dissolved in the oil phase, (ii) duloxetine or its salts also dispersed or dissolved in the oil phase (iii) an omega 3 ethyl ester and (b) an aqueous phase and appropriate pharmaceutically acceptable excipients in the oil phase and aqueous phase.

In another embodiment, the parenteral emulsion formulation comprises (a) an oil phase comprising (i) cannabidiol dispersed or dissolved in the oil phase, (ii) an omega 3 ethyl ester and (b) an aqueous phase comprising acetaminophen and appropriate pharmaceutically acceptable excipients in the oil phase and aqueous phase.

In yet another embodiment, the parenteral emulsion formulation comprises (a) an oil phase comprising (i) cannabidiol dispersed or dissolved in the oil phase, (ii) allopregnanolone also dispersed or dissolved in the oil phase (iii) an omega 3 ethyl ester and (b) an aqueous phase and appropriate pharmaceutically acceptable excipients in the oil phase and aqueous phase.

In yet another embodiment, the parenteral emulsion formulation comprises (a) an oil phase comprising (i) cannabidiol dispersed or dissolved in the oil phase, (ii) brexpiprazole, also dispersed or dissolved in the oil phase (iii) an omega 3 ethyl ester and (b) an aqueous phase and appropriate pharmaceutically acceptable excipients in the oil phase and aqueous phase.

In one embodiment, the parenteral emulsion formulation comprises (a) an oil phase comprising (i) cannabidiol dispersed or dissolved in the oil phase, (ii) an omega 3 ethyl ester and (b) an aqueous phase comprising pregabalin and appropriate pharmaceutically acceptable excipients in the oil phase and aqueous phase.

The samples are analysed using a high-performance liquid chromatographic (HPLC) method. The method uses a reversed-phase Inertsil C18 analytical column (150×4.6 mm I.D., 5 μm particle size) with a mobile phase A of 0.1% orthophosphoric acid and mobile phase B of acetonitrile-water-acetic acid (80:20:1, v/v). An ultraviolet detector operated at 220 nm was used with chromatographic run time of 75 minutes.

The following examples do not restrict the scope of the present invention and are only for the purpose of illustration.

### Example 1

| S. No. | Ingredients | Qty (mg/ml) | Qty (% w/v) |
|---|---|---|---|
| 1. | Cannabidiol | 8 | 0.8 |
| 2. | Omega-3-acid ethyl ester | 60 | 6.0 |
| 3. | Egg lecithin | 10 | 1.0 |
| 4. | PEG-PE | 2 | 0.2 |
| 5. | Glycerol | 20 | 2.0 |
| 6. | Sodium hydrogen carbonate | 0.05 | 0.005 |
| 7. | Milli-Q water | Q.S to 1 mL | Q.S to 100% |

### Manufacturing Procedure:

1. Required quantity of omega 3 ethyl ester was added to manufacturing vessel containing surfactants selected from egg lecithin, PEG-PE, sodium caprylate and dissolved by heating on a water bath at 55° C. (50° C. to 60° C.).
2. Weighed quantity of cannabidiol was added to the above mixture and dissolved by heating on a water bath at 45° C. (40° C. to 50° C.) to give an oil phase.
3. The aqueous phase was prepared by dissolving required quantity of tonicity agent such as glycerol or sodium chloride and required quantities of buffer selected from sodium hydrogen carbonate, sodium hydroxide, sodium citrate and citric acid in 90% of Milli-Q-water by stirring at 400 rpm on a magnetic stirrer maintaining temperature at 55° C. (50° C. to 60° C.).
4. The oil phase was added into the aqueous phase, under homogenization at 8500 rpm by maintaining the product temperature at 45° C. (40° C. to 50° C.) for 15 min and then volume was adjusted up to required level followed by homogenization at 8500 rpm for 15 minutes forming a coarse emulsion.
5. The obtained coarse emulsion was subjected to high pressure homogenization for 3 passes at different pressures i.e. Pass 1 at 10,000 psi, Pass 2 at 18,000 psi and Pass 3 at 18,000 psi followed by subsequent cooling of the product to room temperature to obtain an emulsion.

Physical parameters, particle size distribution and zeta potential (ZP) of the emulsion were determined.

TABLE 2
Particle size data of CBD emulsion

| | | D₁₀ | D₅₀ | D₉₀ | | |
|---|---|---|---|---|---|---|
| HDD(nm) | PDI (%) | (nm) | (nm) | (nm) | SPAN | ZP (mV) |
| 116.59 | 22.43 | 64.40 | 118.24 | 192.66 | 1.13 | −27.23 |

TABLE 3
Physical parameters data of CBD emulsion

| S. No. | Parameter | Result |
|---|---|---|
| 1 | pH | 7.11 |
| 2 | Osmolality (mOsm/Kg) | 236 |
| 3 | Density (g/cm³) | 0.998 |

Further the below examples 2-8 are prepared using manufacturing procedure of Example 1.

### Example 2

| S. No. | Ingredients | Qty (mg/ml) | Qty (% w/v) |
|---|---|---|---|
| 1. | Cannabidiol | 30 | 3.0 |
| 2. | Omega-3-acid ethyl ester | 60 | 6.0 |
| 3. | Egg lecithin | 10 | 1.0 |
| 4. | PEG-PE | 1 | 0.1 |
| 5. | Glycerol | 20 | 2.0 |
| 6. | Sodium hydrogen carbonate | 0.1 | 0.01 |
| 7. | Milli-Q water | Q.s to 1 mL | Q.s to 100% |

TABLE 4
Particle size data of CBD emulsion

| | PDI | | | D₉₀ | | ZP |
|---|---|---|---|---|---|---|
| HDD(nm) | (%) | D₁₀(nm) | D₅₀(nm) | (nm) | SPAN | (mV) |
| 152.17 | 22.43 | 78.94 | 151.0 | 272.67 | 1.30 | −19.53 |

TABLE 5
Physical parameters data of CBD emulsion

| S. No. | Parameter | Result |
|---|---|---|
| 1 | pH | 7.56 |
| 2 | Osmolality (mOsm/Kg) | 252 |
| 3 | Density (g/cm³) | 1.0 |

### Example 3

| S. No. | Ingredients | Qty (mg/ml) | Qty (% w/v) |
|---|---|---|---|
| 1. | Cannabidiol | 8 | 0.8 |
| 2. | Omega-3-acid ethyl ester | 60 | 6.0 |
| 3. | Egg lecithin | 10 | 1.0 |
| 4. | PEG-PE | 2 | 0.2 |
| 5. | Sodium chloride | 7 | 0.7 |
| 6. | Sodium hydrogen carbonate | 0.05 | 0.005 |
| 7. | Milli-Q water | Q.s to 1 mL | Q.s to 100% |

TABLE 6
Particle size data of CBD emulsion

| HDD (nm) | PDI (%) | D₁₀ (nm) | D₅₀ (nm) | D₉₀ (nm) | SPAN |
|---|---|---|---|---|---|
| 130.03 | 15.23 | 79.17 | 120.46 | 180.96 | 0.86 |

TABLE 7
Physical parameters data of CBD emulsion

| S. No. | Parameter | Result |
|---|---|---|
| 1 | pH | 7.08 |
| 2 | Osmolality (mOsm/Kg) | 265 |
| 3 | Density (g/cm³) | 0.997 |

### Example 4

| S. No. | Ingredients | Qty (mg/ml) | Qty (% w/v) |
|---|---|---|---|
| 1. | Cannabidiol | 30 | 3.0 |
| 2. | Omega-3-acid ethyl ester | 60 | 6.0 |
| 3. | Egg lecithin | 10 | 1.0 |
| 4. | PEG-PE | 50 | 5.0 |
| 5. | Glycerol | 20 | 2.0 |
| 6. | Sodium hydrogen carbonate | 0.1 | 0.01 |
| 7. | Milli-Q water | Q.s to 1 mL | Q.s to 100% |

### Example 5

| S. No. | Ingredients | Qty (mg/ml) | Qty (% w/v) |
|---|---|---|---|
| 1. | Cannabidiol | 50 | 5.0 |
| 2. | Omega-3-acid ethyl ester | 60 | 6.0 |
| 3. | Egg lecithin | 10 | 1.0 |
| 4. | PEG-PE | 5 | 0.5 |
| 5. | Glycerol | 20 | 2.0 |
| 6. | Sodium hydrogen carbonate | 0.1 | 0.01 |
| 7. | Milli-Q water | Q.s to 1 mL | Q.s to 100% |

### Example 6

| S. No. | Ingredients | Qty (mg/ml) | Qty (% w/v) |
|---|---|---|---|
| 1. | Cannabidiol | 15 | 1.5 |
| 2. | Omega-3-acid ethyl ester | 40 | 4.0 |
| 3. | Egg lecithin | 10 | 1.0 |
| 4. | PEG-PE | 10 | 1.0 |
| 5. | Glycerol | 20 | 2.0 |
| 6. | Sodium hydrogen carbonate | 0.1 | 0.01 |
| 7. | Milli-Q water | Q.s to 1 mL | Q.s to 100% |

### Example 7

| S. No. | Ingredients | Qty (mg/ml) | Qty (% w/v) |
|---|---|---|---|
| 1. | Cannabidiol | 3 | 0.3 |
| 2. | Omega-3-acid ethyl ester | 60 | 6.0 |
| 3. | Egg lecithin | 10 | 1.0 |
| 4. | PEG-PE | 18 | 1.8 |
| 5. | Sodium chloride | 8 | 0.8 |
| 6. | Sodium hydroxide | pH Adjusted to 7.0 ± 0.5 | |
| 7. | Milli-Q water | Q.s to 1 mL | Q.s to 100% |

### Example 8

| S. No. | Ingredients | Qty (mg/ml) | Qty (% w/v) |
|---|---|---|---|
| 1. | Cannabidiol | 150 | 15.0 |
| 2. | Omega-3-acid ethyl ester | 100 | 10.0 |
| 3. | Egg lecithin | 30 | 3.0 |
| 4. | PEG-PE | 50 | 5.0 |
| 5. | Glycerol | 40 | 4.0 |
| 6. | Sodium hydrogen carbonate | 0.1 | 0.01 |
| 7. | Milli-Q water | Q.s to 1 mL | Q.s to 100% |

### Example 9

| | | F1 | F2 | F3 | |
|---|---|---|---|---|---|
| S. | | Qty | Qty | Qty | Qty |
| No. | Ingredients | (mg/ml) | (mg/ml) | (mg/ml) | (% w/v) |
| 1. | Cannabidiol | 8 | 8 | 8 | 0.8 |
| 2. | Pregabalin | 25 | — | — | 2.5 |
| 3. | Gabapentin | — | 25 | — | 2.5 |
| 4. | Acetaminophen | — | — | 10 | 1.0 |
| 5. | Omega-3-acid ethyl ester | 60 | 60 | 60 | 6.0 |
| 6. | Egg lecithin | 10 | 10 | 10 | 1.0 |
| 7. | PEG-PE | 2 | 2 | 2 | 0.2 |
| 8. | Glycerol | 10 | 10 | 20 | 1.0-2.0 |
| 9. | Sodium hydrogen carbonate | 0.05 | 0.05 | 0.05 | 0.005 |
| 10. | Milli-Q water | Q.s to | Q.s to | Q.s to | Q.s to |
| | | 1 mL | 1 mL | 1 mL | 100% |

### Manufacturing Procedure:

1. Weighed quantity of omega 3 acid ethyl ester was added to manufacturing vessel containing egg lecithin and PEG-PE and dissolved by heating on a water bath at 55° C. (50° C. to 60° C.).
2. Weighed quantity of cannabidiol was added to the above mixture and dissolved by heating on a water bath at 45° C. (40° C. to 50° C.) to give an oil phase.
3. The aqueous phase was prepared by dissolving required quantity of drugs such as pregabalin, gabapentin, acetaminophen, along with glycerol and sodium hydrogen carbonate in 90% of Milli-Q-water by stirring at 400 rpm on a magnetic stirrer maintaining temperature at 55° C. (50° C. to 60° C.).
4. The oil phase was added into the aqueous phase, under homogenization at 8500 rpm by maintaining the product temperature at 45° C. (40° C. to 50° C.) for 15 min and then volume was adjusted up to required level followed by homogenization at 8500 rpm for 15 minutes forming a coarse emulsion.
5. The obtained coarse emulsion was subjected to high pressure homogenization for 3 passes at different pressures i.e. Pass 1 at 10,000 psi, Pass 2 at 18,000 psi and Pass 3 at 18,000 psi followed by subsequent cooling of the product to room temperature to obtain an emulsion.

Physical parameters, particle size distribution and zeta potential (ZP) of the emulsion were determined.

TABLE 8
Particle size data of Example 9

| | HDD | PDI | D₁₀ | D₅₀ | D₉₀ | | ZP |
|---|---|---|---|---|---|---|---|
| Formulations | (nm) | (%) | (nm) | (nm) | (nm) | SPAN | (mV) |
| F1 | 204.95 | 25.87 | 93.80 | 210.17 | 373.07 | 1.34 | — |
| F2 | 185.46 | 23.40 | 100.70 | 188.79 | 311.00 | 1.14 | — |
| F3 | 147.07 | 20.70 | 84.77 | 145.48 | 233.83 | 0.94 | −15.63 |

TABLE 9
Physical parameters of Example 9

| S. No | Parameter | F1 | F2 | F3 |
|---|---|---|---|---|
| 1 | pH | 6.74 | 7.11 | 6.52 |
| 2 | Osmolality (mOsm/Kg) | 279 | 236 | 309 |
| 3 | Density (g/cm³) | 0.996 | 0.998 | 0.996 |

### Example 10

| | | F4 | F5 | F6 | |
|---|---|---|---|---|---|
| S. | | Qty | Qty | Qty | Qty |
| No. | Ingredients | (mg/ml) | (mg/ml) | (mg/ml) | (% w/v) |
| 1. | Cannabidiol | 8 | 15 | 8 | 0.8-1.5 |
| 2. | Duloxetine HCl | 12 | — | — | 1.2 |
| 3. | Allopregnanolone | — | 3 | — | 0.3 |
| 4. | Brexpiprazole | — | — | 4 | 0.4 |
| 5. | Omega-3-acid ethyl | 60 | 40 | 60 | 4-6 |
| | ester | | | | |
| 6. | Egg lecithin | 10 | 10 | 10 | 1 |
| 7. | PEG-PE | 2 | 5 | 2 | 0.2-0.5 |
| 8. | Glycerol | 20 | 20 | 20 | 2.0 |
| 9. | Sodium hydrogen | 1 | 0.1 | 0.05 | 0.005-0.1 |
| | carbonate | | | | |
| 10. | Milli-Q water | Q.s to | Q.s to | Q.s to | Q.s to |
| | | 1 mL | 1 mL | 1 mL | 100% |

### Manufacturing Procedure:

1. Weighed quantity of omega 3 acid ethyl ester was added to manufacturing vessel containing egg lecithin and PEG-PE and dissolved by heating on a water bath at 55° C. (50° C. to 60° C.).
2. Weighed quantity of cannabidiol and drugs such as duloxetine, allopregnanolone, brexpiprazole were added to the above mixture and dissolved by heating on a water bath at 45° C. (40° C. to 50° C.) to give an oil phase.
3. The aqueous phase was prepared by dissolving required quantity of glycerol and sodium hydrogen carbonate in 90% of Milli-Q-water by stirring at 400 rpm on a magnetic stirrer maintaining temperature at 55° C. (50° C. to 60° C.).
4. The oil phase was added into the aqueous phase, under homogenization at 8500 rpm by maintaining the product temperature at 45° C. (40° C. to 50° C.) for 15 min and then volume was adjusted up to required level followed by homogenization at 8500 rpm for 15 minutes forming a coarse emulsion.
5. The obtained coarse emulsion was subjected to high pressure homogenization for 3 passes at different pressures i.e., Pass 1 at 10,000 psi, Pass 2 at 18,000 psi and Pass 3 at 18,000 psi followed by subsequent cooling of the product to room temperature to obtain an emulsion.

Physical parameters, particle size distribution and zeta potential (ZP) of the emulsion were determined.

TABLE 10
Particle size data of Example 10

| | HDD | PDI | D₁₀ | D₅₀ | D₉₀ | | ZP |
|---|---|---|---|---|---|---|---|
| Formulations | (nm) | (%) | (nm) | (nm) | (nm) | SPAN | (mV) |
| F4 | 228.60 | 24.67 | 113.65 | 229.47 | 393.37 | 1.16 | — |
| F5 | 154.32 | 14.23 | 96.84 | 143.37 | 210.83 | 0.79 | −19.63 |

TABLE 11
Physical parameters of Example 10

| S. No | Parameter | F4 | F5 |
|---|---|---|---|
| 1 | pH | 5.85 | 6.62 |
| 2 | Osmolality (mOsm/Kg) | 293 | 259 |
| 3 | Density (g/cm³) | 0.998 | — |

### Example 11

| | | F7 | F8 | |
|---|---|---|---|---|
| | | Qty | Qty | Qty |
| S. No. | Ingredients | (mg/mL) | (mg/mL) | (% w/v) |
| 1. | Cannabidiol | 250 | 250 | 25 |
| 2. | Omega-3 fatty acid | 250 | — | 25 |
| 3. | Omega-3 triglycerides | — | 250 | |
| 4. | Egg lecithin | 50 | 50 | 5.0 |
| 5. | Decanoic acid | 30 | 30 | 3.0 |
| 6. | PEG-PE | 25 | 25 | 2.5 |
| 7. | Glycerol | 20 | 20 | 2.0 |
| 8. | Sodium hydrogen carbonate | 0.05 | 0.05 | 0.005 |
| 9. | Milli-Q water | Q.s to | Q.s to | Q.s to |
| | | 1 mL | 1 mL | 100% |

### Manufacturing Procedure:

1. Weighed quantity of omega-3 component was added to manufacturing vessel containing egg lecithin and PEG-PE and dissolved by heating on a water bath at 55° C. (50° C. to 60° C.).
2. Weighed quantity of cannabidiol was added to the above mixture along with required quantity of decanoic acid and dissolved by heating on a water bath at 45° C. (40° C. to 50° C.) to give an oil phase.
3. The aqueous phase was prepared by dissolving required quantity of glycerol and sodium hydrogen carbonate in 90% of Milli-Q-water by stirring at 400 rpm on a magnetic stirrer maintaining temperature at 55° C. (50° C. to 60° C.).
4. The oil phase was added into the aqueous phase, under homogenization at 8500 rpm by maintaining the product temperature at 45° C. (40° C. to 50° C.) for 15 min and then volume was adjusted up to required level followed by homogenization at 8500 rpm for 15 minutes forming a coarse emulsion.
5. The obtained coarse emulsion was subjected to high pressure homogenization for 3 passes at different pressures i.e. Pass 1 at 10,000 psi, Pass 2 at 18,000 psi and Pass 3 at 18,000 psi followed by subsequent cooling of the product to room temperature to obtain an emulsion.

### Example 12

| | | F9 | F10 | F11 | |
|---|---|---|---|---|---|
| S. | | Qty | Qty | Qty | Qty |
| No. | Ingredients | (mg/ml) | (mg/ml) | (mg/ml) | (% w/v) |
| 1. | Cannabidiol | 6 | 2 | 5 | 0.2-0.6 |
| 2. | Omega-3-acid ethyl | 30 | 30 | 40 | 3-4 |
| | esters | | | | |
| 3. | Egg lecithin | 10 | 10 | 10 | 1.0 |
| 4. | PEG-PE | 15 | 9 | — | 0-1.5 |
| 5. | Sodium caprylate | — | — | 0.5 | 0-0.05 |
| 6. | Glycerol | 20 | — | 20 | 0-2.0 |
| 7. | Sodium citrate | 1 | 1 | — | 0-0.1 |
| 8. | Sodium chloride | — | 8 | — | 0-0.8 |
| 9. | Citric acid | 0.2 | 0.2 | — | 0-0.02 |
| 10. | Milli-Q water | Q.s to | Q.s to | Q.s to | Q.s to |
| | | 1 mL | 1 mL | 1 mL | 100% |

The above formulations (Example 12) are prepared using manufacturing procedure of example 1.

1. A parenteral emulsion formulation of cannabidiol comprising:
(a) an oil phase comprising:
i. cannabidiol in a concentration of about 0.005 mg/ml to about 250 mg/ml, dispersed or dissolved in the oil phase,
ii. an omega 3 component selected from omega 3 fatty acids, omega 3 fatty acid ethyl esters and omega 3 triglycerides and
(b) an aqueous phase and
(c) pharmaceutically acceptable excipients selected from the group comprising surfactants, stabilizers, chelating agents and tonicity agents.

2. The formulation of claim 1, wherein the content of THC is not more than 1% by the weight of the total formulation.

3. (canceled)

4. (canceled)

5. (canceled)

6. The formulation of claim 1, wherein the omega 3 component ranges from about 1_mg/ml to about 500 mg/ml.

7. The formulation of claim 1, wherein the aqueous phase ranges from about 25% w/v to about 95% w/v in the total formulation.

8. The formulation of claim 1, wherein the oil phase ranges from about 5% w/v to about 40% w/v in the total formulation.

9. (canceled)

10. The formulation of claim 1, wherein the content of cannabidiol is not less than 90% after 12 months.

11. (canceled)

12. (canceled)

13. The formulation of claim 1, wherein the aqueous phase comprises one or more buffers selected from the group comprising L-histidine, L-arginine, tris base, meglumine, glycine, sodium succinate, diethanolamine, aspartic acid, glutamic acid, alanine, sodium acetate, sodium ascorbate, sodium citrate, citric acid, succinic acid, triethanolamine, boric acid, sodium hydroxide and sodium hydrogen carbonate.

14. The formulation of claim 1, wherein the stabilizers are selected from the group comprising vitamin E acetate, ascorbyl palmitate, ascorbic acid, monothioglycerol (MTG), citric acid and tartaric acid.

15. The formulation of claim 1, wherein the tonicity agents are selected from the group comprising dextrose, mannitol, glycerol, and sodium chloride.

16. (canceled)

17. (canceled)

18. (canceled)

19. (canceled)

20. A parenteral emulsion formulation of cannabidiol and one or more active ingredients comprising:
(a) an oil phase comprising:
i. cannabidiol and one or more active ingredients selected from allopregnanolone/brexanolone, duloxetine and brexpiprazole dispersed or dissolved in the oil phase,
ii. an omega 3 component selected from omega 3 fatty acids, omega 3 fatty acid ethyl esters and omega 3 triglycerides and
(b) an aqueous phase and
(c) pharmaceutically acceptable excipients-selected from the group comprising surfactants, stabilizers, chelating agents and tonicity agents.

21. The formulation of claim 20, wherein said formulation is used for management of anxiety and depression.

22. A parenteral emulsion formulation of cannabidiol and one or more active ingredients comprising:
(a) an oil phase comprising:
i. cannabidiol dispersed or dissolved in the oil phase,
ii. an omega 3 component selected from omega 3 fatty acids, omega 3 fatty acid ethyl esters and omega 3 triglycerides and
(b) one or more active ingredients selected from pregabalin, acetaminophen and
gabapentin dissolved or dispersed in the aqueous phase and
(c) pharmaceutically acceptable excipients-selected from the group comprising surfactants, stabilizers, chelating agents and tonicity agents.

23. The formulation of claim 22, wherein the said formulation is used for management of pain or inflammation or neuralgia.

24. The formulation of claim 1, wherein the chelating agent is selected from the group comprising 1,4,7,10-tetraazacyclododecane-1,4,7,10-tetraacetic acid (DOTA) and disodium EDTA.
